(19) United States
(12) Patent Application Publication
(10) Pub. No.: US 2024/0428052 A1
(43) Pub. Date: Dec. 26, 2024

(54) METHODS AND SYSTEMS FOR TRAINING MEDICAL MACHINE-LEARNING MODELS

(71) Applicant: Polyview Health, Inc., San Diego, CA (US)

(72) Inventors: Jason Hwang, Henderson, NV (US); Gregory Deocampo, Safety Harbor, FL (US); Dimitri Arges, Henderson, NV (US)

(21) Appl. No.: 18/753,083

(22) Filed: Jun. 25, 2024

Related U.S. Application Data
(60) Provisional application No. 63/509,910, filed on Jun. 23, 2023.
(60) Provisional application No. 63/509,973, filed on Jun. 23, 2023.
(60) Provisional application No. 63/510,006, filed on Jun. 23, 2023.
(60) Provisional application No. 63/510,019, filed on Jun. 23, 2023.
(60) Provisional application No. 63/510,608, filed on Jun. 27, 2023.
(60) Provisional application No. 63/604,930, filed on Dec. 1, 2023.

(51) Int. Cl. G06N 3/0455; G06F 40/35; G06N 3/0475

(52) U.S. Cl. CPC G06N 3/0455; G06F 40/35; G06N 3/0475

(57) ABSTRACT

A generative machine-learning model may be trained using communication session data to tailor a generative model on particular classes of communications. A set of communications sessions may be received with each communication session including, for example, communications between a doctor and a patient. A set of features may be generated by a natural language model from communications extracted from the communication sessions. A subset of the set of communication sessions may be defined based on the set of features. A training dataset may be defined using the subset of the set of communication sessions and used to train a machine-learning model. The machine-learning model can be configured to generate one or more contexts associated with a feature of the set of features. Upon receiving a request associated with a particular, a response may be generated using the machine-learning model and a feature vector derived from the request.

### CROSS-REFERENCE TO RELATED APPLICATIONS

The present patent application claims the benefit of priority to U.S. Provisional Patent Applications 63/509,910, 63/509,973, 63/510,006, and 63/510,019, all of which were filed Jun. 23, 2023; U.S. Provisional Patent Application 63/510,608, filed Jun. 27, 2024; and U.S. Provisional Patent Application 63/604,930, filed Dec. 1, 2023, which are all incorporated herein by reference in their entirety for all purposes.

### TECHNICAL FIELD

This disclosure relates generally to communication session environments, and more specifically, to training and operating machine-learning models on real time communications.

### BACKGROUND

Telehealth can be conducted between a patient and a healthcare provider over a variety of communication channels. These telehealth calls are often facilitated in a standard format or based on third-party's pre-existing systems (e.g., Zoom, Microsoft Teams, Facetime, etc.). Because of the basic nature of current telehealth calls, the calls themselves may not be as beneficial or as efficient as they could be for both the patient and the healthcare provider. For example, it might be more difficult for a patient or healthcare provider to focus on the telehealth call or provide relevant information. As another example, telehealth calls often cause the patient to feel disengaged from the appointment and the healthcare provider, thus creating an environment where the patient might not trust the healthcare provider's advice or might not feel comfortable asking questions.

### SUMMARY

Methods are described herein for training machine-learning models on real time communications. The methods may include receiving a set of communication sessions, wherein each communication session of the set of communication sessions includes communications between a doctor and a patient; extracting the communications from the set of communication sessions; generating a set of features from each communication session of the set of communications session by processing communications of the communication session with a natural language model; defining a subset of the set of communication sessions by filtering one or more communication sessions from the set of communication session based on features extracted from the communications between the doctor and the patient; generating a training dataset from the set of communication sessions; training a machine-learning model using the training dataset, wherein the machine-learning model is configured to generate one or more contexts associated with a feature of the set of features; receiving an identification of a particular feature; generating by the machine-learning model using a vector derived from the particular feature, a context associated with the particular context; and facilitating a presentation of the context.

Systems are described herein for training machine-learning models on real time communications. The systems include one or more processors and a non-transitory computer-readable storage medium storing instructions that, when executing by the one or more processors, cause the one or more processors to perform any of the methods as previously described.

A non-transitory computer-readable medium described herein may store instructions which, when executed by one or more processors, cause the one or more processors to perform any of the methods as previously described.

These illustrative examples are mentioned not to limit or define the disclosure, but to aid understanding thereof. Additional embodiments are discussed in the Detailed Description, and further description is provided there.

### BRIEF DESCRIPTION OF THE DRAWINGS

Features, instances, and advantages of the present disclosure are better understood when the following Detailed Description is read with reference to the accompanying drawings.

FIG. 1 illustrates a block diagram of example communication network configured to facilitate secured communication sessions between users according to aspects of the present disclosure.

FIG. 2 illustrates an example machine-learning training system configured to train generative machine-learning models using training data sets derived from communication sessions according to aspects of the present disclosure.

FIG. 3 illustrates an example operating generative machine-learning models trained on training datasets derived from communication sessions according to aspects of the present disclosure.

FIG. 4 illustrates a flowchart of an example process for generating a review environment of the communication network configured for a particular user device according to aspects of the present disclosure.

FIG. 5 illustrates an example computing device according to aspects of the present disclosure.

### DETAILED DESCRIPTION

Various instances of the disclosure are discussed in detail below. While specific implementations are discussed, it should be understood that this is done for illustration purposes only. A person skilled in the relevant art will recognize that other components and configurations may be used without parting from the spirit and scope of the disclosure.

Methods and systems are described herein for instantiating, training, and operating machine-learning models on real time communications. A communication network may facilitate real-time communication sessions (e.g., communications between two or more users and/or automated services, etc.) that can include text communications, audio communications, video communications, and/or combination thereof. The communication network may aggregate the communication sessions and information associated with the users and/or automated services into a database. The communication sessions and information may be processed and used to instantiate and train machine-learning models usable determine efficacy of communication sessions and/or therapeutics, instantiate automated services configured to expand access to the communication network, etc.

In some examples, a communication session may be a telehealth session including a first user device associated with a patient and a second user device associated with a healthcare provider (e.g., such as, but not limited to, a doctor, nurse, therapist, and/or other healthcare professional). One or more other user devices may also be connected to the communication session. The one or more other user devices may be associated with other users (e.g., such as additional patients, users associated with the patient such as a nurse, additional healthcare providers, etc.). For example, an adult child of an elderly parent may participate in a communication session with the elderly parent and a healthcare provider of the elderly parent.

The communication network may store a record of each communication session. The record may include information associated with the communication session including, but not limited to, communications transmitted by the first user device and/or the second user device (e.g., in an original format or a representation of the communications in a storage format, etc.), any information provided by the first user device and/or the second user device (e.g., such as health records, sensor data, etc.), historical information associated with the patient (e.g., such as, but not limited to, electronic health records, diagnosis, notes generated by the second user device and/or other healthcare providers for which the patient has communicated, combinations thereof, and/or the like), interface data such as data provided through a shared interface of the communication session, metadata, features derived from any of the aforementioned information, combinations thereof, and/or the like.

The communication network may define a set of records for use in training a machine-learning model. The communication network may select records based on one or more common characteristics and/or based on an output intended to be produced by the machine-learning model. For instance, if the machine-learning model is intended for clinical trials (e.g., therapeutic efficacy, therapeutic studies, cohort studies, diagnostics studies, etc.), then the machine-learning model may select records that correspond to communication sessions associated with particular diagnostics and/or therapeutics, etc. Alternatively, the communication network may select communication networks based on a particular healthcare system (e.g., such as a healthcare office, healthcare network, insurance provider, etc.) and/or healthcare provider practice area (e.g., general practitioner, psychology/psychiatry, dermatology, etc.) to train a machine-learning model. Alternatively, the communication network may select records based on time such that the communication network may include select a quantity of the most recently stored records. Alternatively, the communication network may use any of the stored records (e.g., all records, a random quantity of records, etc.) to generate a general machine-learning model.

The communication network may process the set of records to improve the training of the machine-learning model. In some examples, the communication network may first homogenize the set records by translating the data of the set of records into a particular representation such as an alphanumeric representation. The communication network may translate communications that are not already in the particular representation to the particular representation. For instance, speech (or any audio-based communication) may be translated into the alphanumeric representation using a speech-to-text model and visual-based communications may be translated into the alphanumeric representation using an image classifier (e.g., such as a convolutional neural network, or the like, trained to classify gestures). The communication network may then apply one or more additional processes, which may include but are not limited to, removing personal identifiable information, removing device identifiable data (e.g., such as IP addresses, MAC addresses, hardware and/or software profiles, etc.), removing extraneous and/or unnecessary data (e.g., data that may not be usable by the machine-learning model), removing outlier or other data that may impact the training of the machine-learning model (e.g., such as weights, training metrics, accuracy metrics, etc.), applying a dimension reduction algorithm(s) to reduce datasets (e.g., using, for example, principal component analysis, etc.), normalizing data values, adding additional data (e.g., extrapolation, interpolation, procedural generation, etc.) to compensate for aspects of the set of records with too little data, combinations thereof, and/or the like. The additional processes may be executed in any order. The additional processes may be executed in series, in parallel, or any combination thereof. The processed representation of the set of records may be stored in a first data object.

For example, communications exchanged by the first device and the second device during a communication session may include text, speech, and/or gestures. The communication network may translate the communications into an alphanumeric representation (e.g., using a speech-to-text model for speech, an image classifier for gestures, etc.). The alphanumeric representation may be processed using a natural language model. The natural language model may classify aspects of the communications such as meaning and intent, classify aspects of the communication that may be used to identify the first user device or the user thereof, classify aspects of the communication that may be used to identify the second user device or the user thereof, translate the alphanumeric representation into a particular language, etc. The natural language may then remove portions of the alphanumeric representation that may not be relevant or useful such as communications that do not correspond to a purpose of the communication session (e.g., small talk, social communications, etc.), articles and/or other grammatical artifacts, etc. For example, the communication session may be a telehealth session between a healthcare provider and a patient. The natural language model may identify and remove non-healthcare related communications, personal identifiable information, from the record of the communication session. The natural language model may also normalize the remaining alphanumeric representation (e.g., such as by removing unnecessary portions of the alphanumeric representation such as articles and/or punctuation, replacing words with unconjugated variations, reducing words variation by replacing synonyms or similarly defined words with a particularly selected word, etc.). The normalized alphanumeric representation may be stored in a first data object.

The communication network may similarly process other data types such as electronic health records, metadata, notes generated by the second user device (e.g., the healthcare provider), etc. that are included in a record. For instance, each data type may be translated into a common format (e.g., an alphanumeric format, etc.), and processed by a data model (e.g., an algorithm, machine-learning model, etc.) trained to classify and remove the portions of the data of the data type that may identify a user, that are unrelated to the purpose of the communication session, etc. For graphical data (e.g., such as video, images, graphics, etc.), the communication network may use a convolutional neural network or other machine-learning model to derive features from the graphical data. The features may include, but are not limited to, an identification of the graphical data (e.g., a classification of a video frame, image, or graphic), a semantic definition of the graphical data, an identification of any objects and/or users depicted in the graphical data, watermark and/or cryptographic data (e.g., for data security), data values depicted by or derivable from the graphical data (e.g., such as alphanumeric data from graphs, charts, etc.), combinations thereof, and/or like. The processed representation of the other data types may be stored in a second data object (e.g., which may be the first data object or another data object).

The communication network may define a training dataset from the processed set of records to train a machine-learning model. The machine-learning model may be a untrained machine-learning model, a partially-pretrained machine-learning model, a pretrained machine-learning model, etc. Examples of machine-learning models may include, but are not limited to, deep learning networks, neural networks, transformers (such as generative pre-trained transformers (GPT), Bidirectional Encoder Representations from Transformers (BERTs), text-to-text-transfer-transformer (T5), etc.), classifier, variational autoencoders, generative adversarial networks (GANs), recurrent gated units (GRUs), combinations thereof, or the like. In some instances, the machine-learning model may be generative pre-trained transformer type model. In other instances, the machine-learning model may be an ensemble model comprised of one or more interconnected machine-learning models (e.g., of a same type or one or more types).

The machine-learning model may be trained using supervised learning, unsupervised learning, semi-supervised learning, transfer learning, metalearning, reinforcement learning, combinations thereof, or the like using the training dataset. In some instances, such as when the training dataset is too small or is limited (e.g., particular data are over-represented in the training data and/or particular data are under-represented in the training data), additional data may be added to the training data. The additional data may include manually generated data, procedurally generated data, combinations thereof, and/or the like. Metadata may also be added to the training data to enable particular types of learning algorithm. For instance, for supervised learning, labels (as metadata) may be added to the training data. The labels may be generated manually (e.g., via user input, etc.), by an already trained instance of the machine-learning model, by a generative adversarial network, combinations thereof, and/or the like. The machine-learning model may be trained over a predetermined time interval, for predetermined quantity of iterations, and/or until one or more accuracy metrics are reached (e.g., such as, but not limited to, accuracy, precision, area under the curve, logarithmic loss, F1 score, a longest common subsequence (LCS) such as ROUGE-L, Bilingual evaluation Understudy (BLEU) mean absolute error, mean square error, or the like.

The trained machine-learning model may be configured to generate information associated with the communication sessions used for training. In some instances, as the communication session facilitated additional communication sessions, the communication sessions may be processed (using any of the aforementioned processes) and used to further train and/or reinforce the training of the machine-learning model. In those instances, the machine-learning model may be continually retrained to continuously improve the accuracy and usability of the machine-learning model.

The trained machine-learning model may be configured to recall aspects of previous communication sessions, generate analytics associated with sets of communication sessions (e.g., such as, but not limited to, trends, single or multivariate analysis, root cause analysis, etc.), define study protocols, determine efficacy of treatments or medical protocols, generate processes that may improve the communication session (e.g., define topics, conversation, statements, etc. that may improve information provided by healthcare providers and/or patients), modify the communication network (e.g., to improve communication network, interfaces generated by or associated with the communication network, environments generated by or associated with the communication network, etc.), query for particular communications provided during a communication session, combinations thereof, and/or the like.

For example, the communication network may generate a feature vector to be input to the machine-learning model. In some instances, the feature vector may be automatically generated by the communication network based on one or more communication sessions facilitated by the communication network. For example, the communication network may include thresholds associated with various aspects of the communication sessions that may trigger execution of the machine-learning model. The thresholds may include a frequency in which various functions of the communication network are accessed by user devices and/or users thereof, a network or input latency, contents of communications (e.g., indicating functions that are liked or disliked, criticized, etc.) from communication sessions or outside of communication sessions, combinations thereof, and/or the like. The communication network may execute the machine-learning model to generate modifications to the communication network, communication session, interfaces, environments, combinations thereof, and/or the like. For example, the modifications may also to improve access and operability of the communication network, communication session, interfaces, environments, combinations thereof, and/or the like by rearranging particular icons, functions, menus, interfaces, etc. based on frequency of use, importance, and/or any other metric defined by the machine-learning model and/or user input). The modifications may also to improve access and operability of the communication network, communication session, interfaces, environments, combinations thereof, and/or the like by removing particular icons, functions, menus, interfaces, etc. such as those that are infrequently accessed, etc.

In other instances, the feature vector may be generated by the communication network based on user input received from one or more users. The user input may be natural language question or statement, one or more commands, a query, a data object, combinations thereof, and/or the like. The user input may request particular information (e.g., such as analytics associated with communication, trends, a single variate analysis, a multivariate analysis, a query, combinations, thereof, or the like). In some examples, the input may include an identification of a representation of the output of the machine-learning model, such as, but not limited to, a chart, a graph, an image, a video segment, an infographic, an audio segment, alphanumeric text, a data object (e.g., accessible by the communication network), an interface (e.g., accessible or presentable by the communication network, the first user device, the second user device, the device providing the user input, etc.), combinations thereof, and/or the like.

Although described in connection with healthcare services, the training of the machine-learning model may use any conversation data associated with particular category or classification to generate a machine-learning model trained on a particular category or classification of communications. The trained machine-learning model may be configured to generate outputs that may be tailored to the particular category and/or classification.

In an illustrative example, a computing device (e.g., of a communication network, etc.) may receive a set of communication sessions. Each communication session may be a telehealth session and include communications between a healthcare provider and a patient. The computing device may receive the set of communication sessions from local or remote memory, one or more other instances of a communication network, combinations thereof, and/or the like. For instance, the computing device may receive one or more communication sessions facilitated by the computing device and/or one or more communication sessions from another communication network. Each communication session may include a record of the telehealth session including, but not limited to, communications exchanged during the telehealth session (e.g., text, audio segments, video segments, combinations thereof, and/or the like), notes generated by the healthcare provider and/or the patient, metadata, data and/or media provided via a shared interface of the communication session, combinations thereof, and/or the like.

The computing device may extract the communications from the set of communication sessions. In some instances, the computing device may extract the communications from each communication session of the set of communication sessions. In other instances, the computing device may extract particular communications from each communication session of the set of communication sessions or extract the communications from a particular subset of communication sessions of the set of communication session. The particular communications and/or the particular subset of communication sessions may be selected based on one or more parameters such as an identification of a meaning and/or intent of the communication, an identification of a particular healthcare system associated with the communication or communication session (e.g., such as a healthcare office, healthcare network, insurance provider, etc.), an identification of a healthcare provider practice area (e.g., general practitioner, psychology/psychiatry, dermatology, etc.) associated with the communication or communication session, an identification of a purpose of the healthcare session (e.g., check up or physical, symptoms, diagnosis, etc.), combinations thereof, and/or the like.

The computing device may generate a set of features from each communication session of the set of communications session by processing the extracted communications with a natural language model. The extracted communications may include natural language text, speech, and/or gestures. The communication network may translate the communications into an alphanumeric representation (e.g., using a speech-to-text model for speech, an image classifier for gestures, etc.). The natural language model may classify aspects of the communications such as meaning and intent, classify aspects of the communication that may be used to identify the patient or the healthcare provider, translate the alphanumeric representation into a particular language, combinations thereof, and/or the like. The natural language model may then remove portions of the alphanumeric representation that may not be relevant or useful such as communications that do not correspond to a purpose of the communication session (e.g., small talk, social communications, etc.), articles and/or other grammatical artifacts, etc. The natural language model may also normalize the remaining alphanumeric representation (e.g., such as by removing unnecessary portions of the alphanumeric representation such as articles and/or punctuation, replacing words with unconjugated variations, reducing words variation by replacing synonyms or similarly defined words with a particularly selected word, etc.). The normalized alphanumeric representation may be stored in a first data object.

In some examples, the computing device may process other data of a communication session such as, but not limited to, data associated with devices operated by the healthcare provider and/or the patient, metadata, objects transmitted via a shared interface of the communication session, combinations thereof, and/or the like. The other data may be processed by one or more machine-learning models, the natural language model, one or more algorithms, user input, any of the aforementioned processing techniques, combinations thereof, and/or the like. The processed other data may be stored in a second data object that may be combined with the first data object or stored in association with the first data object.

The computing device may define a subset of the set of communication sessions by filtering, from the first data object and/or the second data object, data of one or more communication sessions from the set of communication sessions based on features associated with the communications extracted by the natural language model. In some instances, the computing device may define a subset of the set of communication sessions when the computing devices extracts communications from each of the communication sessions. In other instances, the computing device may define a subset of the set of communication sessions in addition to the selective extraction of communications as described above. The features may include, but are not limited to, a practice area of the healthcare provider associated with the communication session, purpose of the communication session (e.g., checkup, treatment, symptoms, diagnosis, diagnostics or testing, therapy, combinations thereof, and/or the like), an identification of a diagnosis, an identification of a healthcare system, demographic information associated with the patient and/or healthcare provider, a frequency of communication sessions between the healthcare provider and the patient, a frequency of communication sessions associated with the healthcare provider, a frequency of communication sessions associated with the patient, combinations thereof, and/or the like.

The computing device may generate a training dataset from the set of communication sessions. Generating a training dataset may include aggregating the remaining data from the first data object and the second data object. The computing device may evaluate the training dataset to determine if additional data is needed for the training dataset to be usable. In some instances, such as when the training dataset is too small or is limited (e.g., particular data are over-represented in the training data and/or particular data are under-represented in the training data), additional data may be added to the training data. The additional data may include manually generated data, procedurally generated data, combinations thereof, and/or the like. Metadata may also be added to the training data to enable particular types of learning algorithm. For instance, for supervised learning, labels (as metadata) may be added to the training data. The labels may be generated manually (e.g., via user input, etc.), by an already trained instance of the machine-learning model, by a generative adversarial network, combinations thereof, and/or the like.

The computing device may train a machine-learning model using the training dataset. The machine-learning model may be a generative machine-learning model generate one or more contexts associated with a feature of the set of features extracted by the natural language model. A context may correspond to a trend, query response, analytic, efficacy of a treatment, single variate analysis, multi variate analysis, modification (e.g., to the computing device, a communication network, a communication session, etc.), a recommended improvement, a classification, a sentiment, an intent, combination thereof, and/or the like. Examples of machine-learning models may include, but are not limited to, deep learning networks, neural networks, transformers (such as generative pre-trained transformers (GPT), Bidirectional Encoder Representations from Transformers (BERTs), text-to-text-transfer-transformer (T5), etc.), classifier, variational autoencoders, generative adversarial networks (GANs), recurrent gated units (GRUs), combinations thereof, or the like. In some instances, the machine-learning model may be generative pre-trained transformer type model. In other instances, the machine-learning model may be an ensemble model comprised of one or more interconnected machine-learning models (e.g., of a same type or one or more types).

The machine-learning model may be trained using supervised learning, unsupervised learning, semi-supervised learning, transfer learning, metalearning, reinforcement learning, combinations thereof, or the like using the training dataset. The machine-learning model may be trained over a predetermined time interval, for predetermined quantity of iterations, and/or until one or more accuracy metrics are reached (e.g., such as, but not limited to, accuracy, precision, area under the curve, logarithmic loss, F1 score, a longest common subsequence (LCS) such as ROUGE-L, Bilingual evaluation Understudy (BLEU) mean absolute error, mean square error, or the like.

The computing device may receive a request associated with a feature. The request may include a query, a natural language statement, a command, an image, an audio segment, a video segment, combinations thereof, and/or the like. The computing device may generate a feature vector from the request and based on the feature. The feature vector may include the request (e.g., in a same representation as received, translated into a particular representation, translated into a particular sequence, and/or the like).

The computing device may generate, using the machine-learning model and a feature vector derived from the feature, a context associated with the particular feature. For example, the request may correspond to a study associated with a treatment protocol provided by the healthcare provider to one or more patients. The context may include trends, analytics, single variate and/or multivariate analysis, combinations thereof, and/or the like providing characteristics of the treatment protocol such as efficacy of the treatment protocol, an identification of outliers in the treatment (e.g., such as new or rarely identified side effects, etc.), identification of patient sentiment of the treatment protocol, an identification of recommended follow up studies and/or treatments, root cause analysis, charts, graphs, infographics, combinations thereof, and/or the like.

The computing device may facilitate a presentation of the context. The presentation of the context may include presenting the context via a graphical user interface, a video segment, an audio segment (e.g., using narration derived from a text-to-speech model and/or a user), combinations thereof, and/or the like. In some instances, the context may be used to modify the computing device, subsequent communication sessions facilitated by a communication network, the machine-learning model (e.g., via reinforcement learning, etc.), combinations thereof, and/or the like.

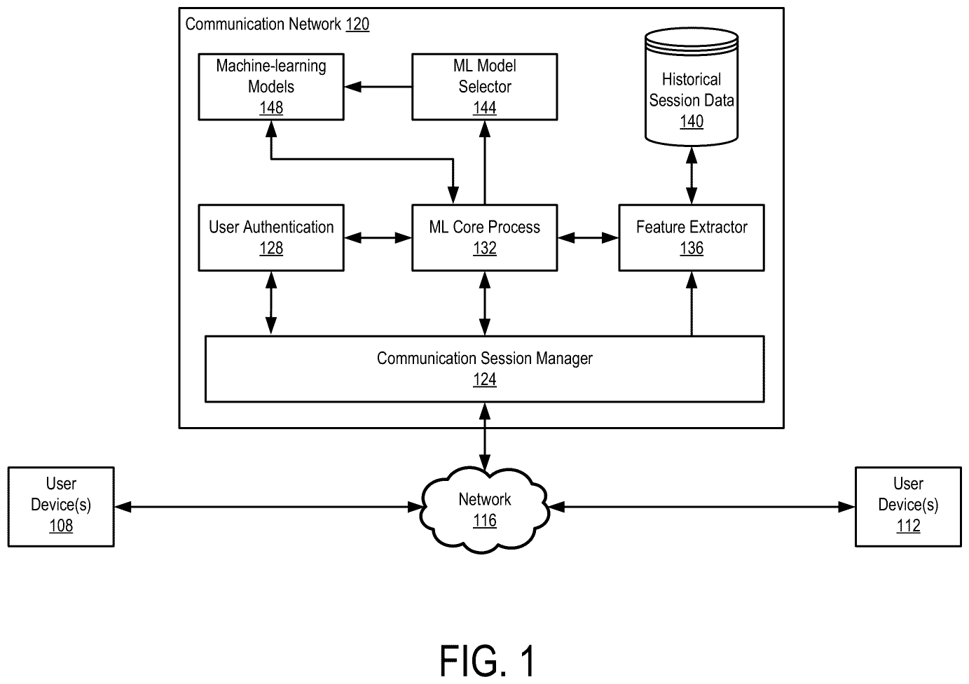

FIG. 1 illustrates a block diagram of example communication network configured to facilitate secured communication sessions between users according to aspects of the present disclosure. Communication network 120 may facilitate communication sessions between a first user device operated by a patient (e.g., user device 108) and a second user device operated by a healthcare provider (e.g., user device 112). Communication network 120 may enable one or more other devices associated with the first user device (e.g., such as nurses, aides, social workers, parents, adult children, etc.) and/or the second user device (e.g., nurses, assistants, other doctors, administrators, etc.) to connect to the communication session.

Communication network 120 may include one or more processing devices (e.g., computing devices, mobile devices, servers, databases, etc.) configured to operate together to provide the services of communication network 120. The one or more processing devices may operate with a same local network (e.g., such as a local area network, wide area network, mesh network, etc.) or may be distributed processing devices (e.g., such a cloud network, distributed processing network, or the like). User device 108 and user device 112 may connect to communication network 120 directly or through one or more intermediary networks 116 (e.g., such as the Internet, virtual private networks, etc.).

The first user device or the second user device may request a new communication session using communication session manager 124. The request may include an identification of one or more user devices are authorized to connect to the communication session. The request may also include other parameters such as user profile data (associated with the first user device or the second user device, such as, but not limited to an identification of a user of the user profile, a user identifier, user devices operated by the user), a purpose for establishing the communication session, a start time of the communication session, an expected duration of the communication session, settings of the communication session (e.g., audio channel settings, video channel settings, collaborative window settings, wrapper settings, etc.), combinations thereof, or the like.

Communication session manager 124 may then instantiate a new communication session for the first user device and/or the second user device. The new communication session may include one or more environments for the user devices connected to the communication session. The environment may include user interfaces, wrappers, resources, application programming interfaces, etc. configured to extend the functionality of the communication session. The communication session manager 124 using ML core process 132 may provision one or more machine-learning models to enable any of the extended functionality. The one or more machine-learning models may be configured to provide natural language processing (e.g., such as a large language model, bi-directional transformers, zero/few shot learners, deep neural networks, etc.), content generation (e.g., using large language models, deep neural networks, generative adversarial networks, etc.), single variate or multivariate classifiers (e.g., k-nearest neighbors, random forest, logarithmic regression, decision trees, support vector machines, gradient decent, etc.), image processing (e.g., using deep neural networks, convolutional neural networks, etc.), sequenced data processors (e.g., such as recurrent neural networks, etc. capable of processing datasets organized according to a taxonomic sequence), and/or the like. The one or more machine-learning models may be configured to process natural language communications (e.g., such as gestures, verbal, textual, etc.) to provide real-time translations and/or transcriptions, generate natural language communication capable of autonomous communication (e.g., a communication bot) or content generation (e.g., such generating natural language responses to requests for information, etc.), user authentication (e.g., to ensure users connected to the communication session are authorized to do so), and/or the like.

Communication session manager 124 may authenticate each user device that connects to the new communication session using user authentication 128. User authentication 128 may ensure that the user of a connected user device corresponds to an authorized user. To avoid exposing personal identifiable information or medical information, user authentication 128 may compare abstracted features associated with the user to corresponding abstracted features associated with an authorized user. In some instances, the abstracted features may include an abstracted representation of a username, password, token, public/private key, and/or the like. Communication session manager 124 may distribute passwords, tokens, public/private keys, and/or the like with an invitation to connect to the new communication session. In some instances, the abstracted features may include biometric features of a user of a user device to be authenticated such as physical features, vocal features, and/or the like. For example, using monocular depth estimation facial features can be extracted based on a relative distance of a representation of the facial feature (e.g., in a video frame) from the camera that captured the representation. The relative distances may be used to determine if the user a user device corresponds to a known, authenticated user by comparing the relative distances to stored relative distances.

User authentication 128 may obtain one or more video frames and/or one or more audio segments received from a user device to be authenticated. The one or more video frames may be generated using a camera of the user device and include a representation of a user of the user device. The audio segments may include a representation of a voice of the user. User authentication 128 may transmit a request to machine-learning (ML) core process 132 to process the video and/or audio. For example, using a first machine-learning model of machine-learning models 148, a depth map may be generated using a video frame including a representation of a user. The depth map may include a distance value for each pixel of the video frame corresponding to a predicted distance of a point in the environment represented by the pixel from the camera that captured the video frame. User authentication 128 may use the depth map to distinguish pixels corresponding to the user from pixels corresponding to the background (e.g., based on the pixels representing the user being generally closer than pixels representing the background). User authentication 128 may then determine relative differences in distances between one or more pixels to determine a relative depth of one or more facial features. The relive differences in distances may be abstracted features that may be used to determine whether the user represented by the video data is authenticated by comparing the abstracted features to abstracted features of an authenticated user.

Using a second machine-learning model, user authentication 128 may process audio segments including a representation of the user's voice. The second machine-learning model may process the audio segments to derive abstracted features associated with the audio segments, the second machine-learning model may be configured to identify pitch, tone, speech velocity, pause frequency and length, diction, accent, language, etc.) of the audio segment represented as a sequence of numerical values. The abstracted features can be compared to historical abstracted features of an authenticated user to determine if the user associated with the abstracted features is the same user as the user associated with the historical abstracted features.

Communication session manager 124 may pass communications extracted over the communication session to ML core process 132 process the communications using the one or more machine-learning models. ML core process 132 may monitor one or more machine-learning models configured to provide the services of the communication network. ML core process 132 may train new machine-learning models, retrain (or reinforce) existing machine-learning models, delete machine-learning models, and/or the like. Since ML core process 132 manages the operations of a variety of machine-learning models, each request to ML core process 132 may include an identification of a particular machine-learning model, a requested output, or the like to enable ML core process 132 to route the request to an appropriate machine-learning model or instantiate and train a new machine-learning model. Alternately, ML core process 132 may analyze data to be processed that is included in the request to select an appropriate machine-learning model configured to process data of that type.

If ML core process 132 cannot identify a trained machine-learning model configured to process the request, then ML core process 132 may instantiate and train one or more machine-learning models configured to process the request. Machine-learning models may be trained to process a particular input and/or generate a particular output. ML core process 132 may instantiate and train machine-learning models based on the particular data to be processed and/or the particular output requested. For example, user sentiment analysis (e.g., user intent, etc.) may be determined using a natural language processor and/or a classifier while image processing may be performed using a convolutional neural network.

ML core process 132 may select one or more machine-learning models based on characteristics of the data to be processed and/or the output expected. ML core process 132 may then use feature extractor 136 to generate training datasets for the new machine-learning models (e.g., other than those models configured to perform feature extraction such as some deep learning networks, etc.). Feature extractor 136 may define training dataset using historical session data 140. Historical session data 140 may store features from previous communication sessions. In some instances, the previous communication sessions may not involve the user of the first user device or the user of the second user device. Previous communication sessions may include manually and/or procedurally generated data generated for use in training machine-learning models. Historical session data 140 may not store any information associated with healthcare providers or patients. Alternatively, historical session data 140 may store features extracted from communication session involving the user of the first user device, the user of the second user device, and/or other patients and/or other healthcare providers.

Feature extractor 136 may extract features based on the type of model to be trained and the type of training to be performed (e.g., supervised, unsupervised, etc.) from historical session data 140. Feature extractor 136 may include a search function (e.g., such as procedural search, Boolean search, natural language search, large language model assisted search, or the like) to enable ML core process 132, an administrator, or the like to search for particular datasets within historical session data 140 to improve the data selection for the training datasets. Feature extractor 136 may aggregate the extracted features into one or more training datasets usable to train a respective machine-learning model of the one or more machine-learning models. The training datasets may include training datasets for training the machine-learning models, training datasets to validate an in-training or trained machine-learning model, training datasets to test a trained machine-learning model, and/or the like. The one or more training datasets may be passed to ML core process 132, which may manage the training process.

Feature extractor 136 may pass the one or more training datasets to ML core process 132 and ML core process 132 may initiate a training phase for the one or more machine-learning models. The one or more machine-learning models may be trained using supervised learning, unsupervised learning, self-supervised learning, or the like. The one or more machine-learning models may be trained for a predetermined time interval, a predetermined quantity of iterations, until one or more target accuracy metrics have exceeded a corresponding threshold function (e.g., accuracy, precision, area under the curve, logarithmic loss, F1 score, weighted human disagreement rate, cross entropy, mean absolute error, mean square error, etc.), user input, combinations thereof, or the like. Once trained, ML core process 132 may validate and/or test the trained machine-learning models using additional training datasets. The machine-learning models may also be trained at runtime using reinforcement learning.

Once the machine-learning models are trained, ML core process 132 may manage the operation of the one or more machine-learning models (stored with other machine-learning models in machine-learning models 148) during runtime. ML core process 132 may direct feature extractor 136 to define feature vectors from received data (e.g., such as the video data, audio segments from the first user device or the second user device, content of a collaborative window such as collaborative window 204, etc.). In some instances, ML core process 132 may facilitate generation of a feature vector each time there is a change in the communication channel (e.g., a change in video from a user device, an audio segment is transmitted over the communication channel, content is added or removed from a user interface of the communication session (e.g., such as patient interface 200, provider interface 232, temporary environment 252, or any other interface displayed to a user device), content is modified from a user interface of the communication session (e.g., such manipulating an image, etc.), a timestamp relative to a start time of the communication session, or the like. ML core process 132 may continually execute the one or more machine-learning models to generate corresponding outputs. ML core process 132 may evaluate the outputs to determine whether to manipulate a user interface of the communication session based on the output (e.g., post automatically generated content, modify a word wall weights or words, add/remove suggested questions, initiate an automated conversation with a bot, provide information associated with keywords spoken during the communication session, etc.).

For example, ML core process 132 may detect a new audio segment communicate over the communication session. ML core process 132 execute a machine-learning model (e.g., such as a recurrent neural network) to process the audio segment to determine the words within the audio segment (if any) and a sentiment (e.g., a predicted meaning of the individual words or the words as a whole). ML core process 132 may execute another machine-learning model (e.g., such as a classifier, a large language model and/or transformer, a generative adversarial network, etc.), to generate content corresponding to the words and/or sentiment that can be provided to a user device. For instance, the words may include “My knee is swollen and painful” with a sentiment of “symptoms.” The other machine-learning model may process the words and sentiment to generate content for patient interface 200 such as information about ailments associated with knee pain and knee swelling, home treatments that may alleviate symptoms and/or improve mobility, possible questions that can be asked to the healthcare provider, etc. ML core process 132 may also use the other machine-learning model to generate content for provider interface such as symptoms, suggested follow up questions regarding the degree of swelling or the intensity of the pain, links for additional information associated with knee pain or knee swelling, links associated with ailments associated with knee pain or knee swelling, etc.

ML core process may direct feature extractor 136 to define other feature vectors to process other data using machine-learning models of machine-learning models 148 in parallel with the aforementioned machine-learning models to provide other resources of communication network 120. ML core process 132 may execute any number of machine-learning models in parallel to provide the functionality of a communication session.

Communication session manager 124 may update the interfaces (e.g., patient interface 200, provider interface 232, temporary environment 252, and/or any other interface presented to user devices during a communication session) in real-time. Content may be received from a particular user device associated with a collaborative window (that may be viewable by all user devices connected to the communication session) via a drag and drop over the collaborative window, an upload link, or the like. Communication session manager 124 may process the received content into a format that can be embedded into the collaborative window and update the collaborative window (or the entire interface) enabling other user devices connected to the communication session to view the content in a same manner as provided by the particular user device. Communication session manager 124 may also receive outputs from machine-learning models 148 via ML core process 132 and determine whether to update the interfaces using the output and how to update the interfaces using the output (e.g., where to present generated content within the interface, what fonts and font sizes to present generated content, whether generated content is to expire after a time interval and be removed from the interface, whether the content is related to and should presented proximate to other content, etc.). Communication session manager 124 may continuously update and the interfaces to present a dynamic, collaborative interface to each user device through the communication session. When the communication session terminates, communication session manager 124 may store the interfaces with an identifier associated with the communication session for further processing and/or replay by a user device of the communication session.

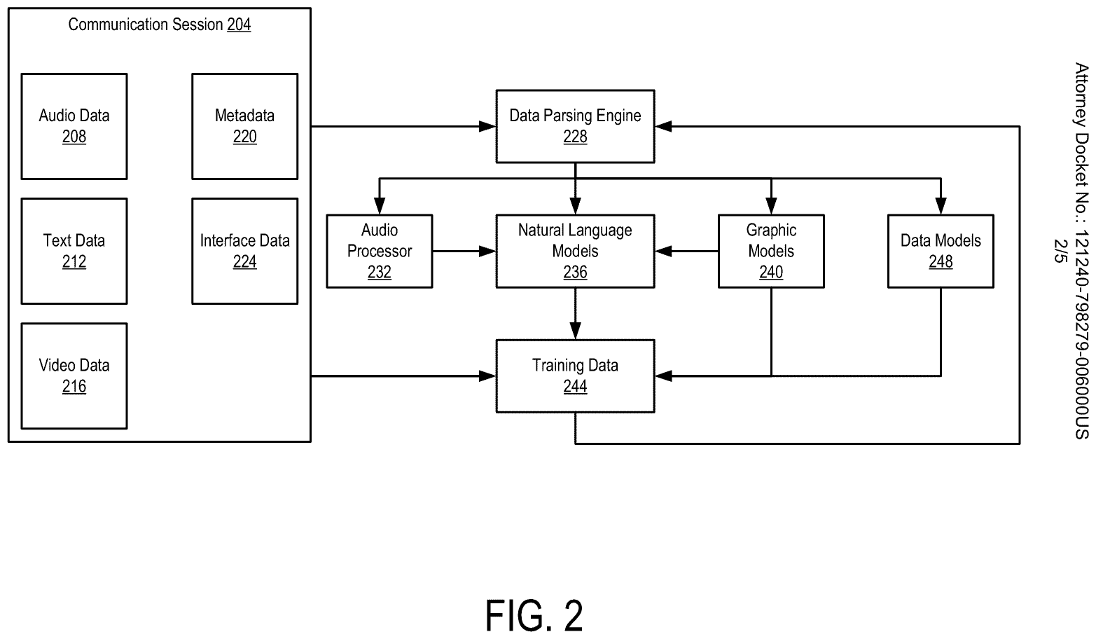

FIG. 2 illustrates an example machine-learning training system configured to train generative machine-learning models using training data sets derived from communication sessions according to aspects of the present disclosure. A communication network (e.g., such as communication network 120 of FIG. 1), may facilitate a communication session between one or more first users (e.g., a healthcare provider, users associated with the healthcare provider, etc.) and one or more second users (e.g., patients, users associated with patients, etc.). The communication session may enable an exchange of communications for a particular purpose such as (in this example) a telehealth session. The communication session may be usable to facilitate communication sessions for other purposes.

Communication session 204 may be a record of a communication session facilitated by the communication network. Communication session 204 may include information associated with the communication session including, but not limited to, audio data 208 (e.g., including audio segments of transmitted by the one or more first users and/or the one or more second users, transcripts of the audio segments, metadata associated with the audio segments, combinations thereof, and/or the like, text data 212 (e.g., including any alphanumeric text transmitted by the one or more first users and/or the one or more second users during the communication session, etc.), video data 216 (e.g., including video segments transmitted by the one or more first users and/or the one or more second users, transcripts of the video segments such as any gesture-based communications, metadata (e.g., frame rate, resolution, visual artifacts, watermarks, etc.), combinations thereof, and/or the like), metadata (e.g., such as, but not limited to, historical information associated with the one or more first users such as any health related information and/or electronic health records, information associated previous sessions between the one or more first users and the one or more second users, information associated with insurance of the one or more first users, historical information associated with the one or more second users, information provided by the one or more first users such as notes, an identification of topics discussed during the communication session, an identification of symptoms discussed during the communication session, an identification treatments discussed during the communication session, an identification diagnosis discussed during the communication session, combinations thereof, information provided to the one or more first users by the one or more second users, and/or the like), interface data 224 (e.g., any information or media provided by the one or more first users and/or the one or more second users over the shared interface provided by the communication session, etc.), combinations thereof, and/or the like.

Communication session 204 may be augmented with additional data associated with the communication session including any information transmitted or derived from information transmitted by the one or more first users and/or the one or more second users, information associated previous communication session, information associated with the communication network, combinations thereof, and/or the like.

Communication session 204 may be processed using data parsing engine 208. Data parsing engine 208 may define a processing pipeline for each data type of communication session 204. The processing pipelines may be defined at runtime (e.g., dynamically, etc.) based on an analysis of the contents of a communication session. For example, data parsing engine 232 may translate audio segments into text using a speech-to-text model of audio processor 232, processes the text using natural language models 235, and output the result to training data 244. Data parsing engine 228 may pass video data 216 (and/or other graphical data of communication session 204) to graphic models 248 to derive features from the video data. The features associated with communications (e.g., gestures, sign language, etc.) may be passed to natural language models 236 for further processing and features that are not associated with communications may be passed directly to training data 244. Data parsing engine 228 may pass any data that is not a communication, audio segment, or graphical to data models 244 (e.g., such as charts, electronic health records, etc.)

Audio processor 232 may include one or more machine-learning models, algorithms, interfaces, and/or protocols usable to process audio-segments. In some instances, audio processor 232 may include a speech-to-text machine-learning model (e.g., such a recurrent neural network configured to process time-series information), which may output text representations of speech. Audio processor 232 may also include other models and algorithms to modify the audio segments to improve the audio processing by the speech-to-text machine-learning model. The other models and algorithms may include filters and/or the like configured to remove noise and improve the frequency bands associated human speech.

Natural language models 236 may include one or more machine-learning models, algorithms, interfaces, and/or protocols usable to process natural language text. The text may be generated by the one or more first users and/or the one or more second users, by the audio processor 232, by graphic models 240, combinations thereof, and/or the like. Natural language models 236 may determine a meaning of words, phrases, and/or sentences; determine an intent of a user that generated the words, phrases, and/or sentences; converting words, phrases, and/or sentences into a generic representation (e.g., replacing words with more frequently used synonyms, removing articles and/or other grammatical components, etc.); translating words, phrases, and/or sentences into a common language, combinations thereof, and/or the like.

Graphic models 240 may include one or more machine-learning models, algorithms, interfaces, and/or protocols usable to process graphical content such as, but not limited to, images, graphs, infographics, video, etc. In some instances, graphic models 240 may include one or more convolutional neural networks configured to classify images and video frames to identify objects that may be relevant to the communication session (e.g., medical devices, sensors, etc.), gestures (e.g., such as non-verbal communications, sign language, etc.), a status of the user depicted in an image or video (e.g., such as general health, fatigue, complexation, age, pain level, heart rate, etc.) combinations thereof, and/or the like.

Data models 248 may include one or more machine-learning models, algorithms, interfaces, and/or protocols usable to process data (e.g., any data that is not processed by audio processor 232, natural language models 236, graphic models 240, and/or the like.

The output from natural language models 236, graphic models 240, and/or data models 244 may be received by training data 244. Training data 244 may be a dataset of the processed representation of one or more communication sessions usable to train a generative machine-learning model. The processing of communication session 204 may be iterative. For instance, once the audio segments, video segments, text-based communications, and data have been processed, training data 244 may pass the processed representations to data parsing engine 228 for additional processing. Examples of additional processes may include but are not limited to, removing personal identifiable information, removing device identifiable data (e.g., such as IP addresses, MAC addresses, hardware and/or software profiles, etc.), removing extraneous and/or unnecessary data (e.g., data that may not be usable by the machine-learning model), removing outlier or other data that may impact the training of the machine-learning model (e.g., such as weights, training metrics, accuracy metrics, etc.), applying a dimension reduction algorithm(s) to reduce datasets (e.g., using, for example, principal component analysis, etc.), normalizing data values, adding additional data (e.g., extrapolation, interpolation, procedural generation, etc.) to compensate for aspects of the set of records with too little data, combinations thereof, and/or the like. The additional processes may be executed in any order. The additional processes may be executed in series, in parallel, or any combination thereof. After each iteration of processing the data may be store in training data 244. If no further processing of the data is needed, then data parsing engine 228 may begin processing another communication session.

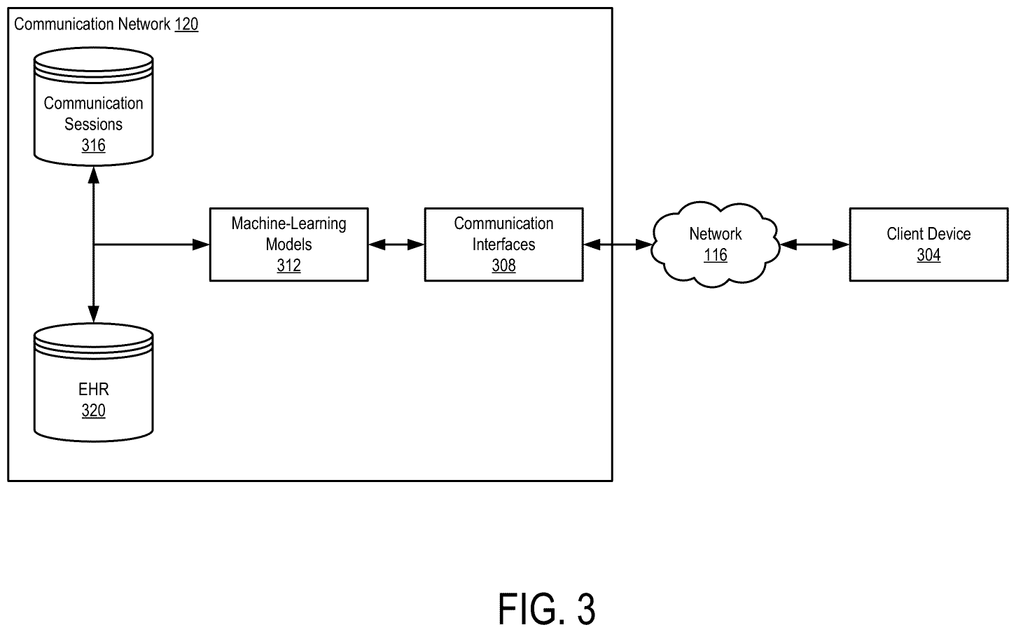

FIG. 3 illustrates an example operating generative machine-learning models trained on training datasets derived from communication sessions according to aspects of the present disclosure. Communication network 120 may include one or more machine-learning models trained to generate information associated with communication session facilitated by communication network 120. Each machine-learning model may be trained using a selected set of communication sessions to tailor the accuracy and efficiency of the machine-learning model. Communication network 120 may be configured to instantiate and train new machine-learning models based on particular sets of features. For instance, client device 304 may transmit a request over network 116 to communication network 120. Client device 304 may be operated by a patient, a healthcare provider, a user of a healthcare system, a user of an insurance provider, a user of a pharmaceutical manufacturer, scientist, and/or any other user authorized to extract information associated communication network 120.

Communication interfaces 308 of communication network 120 may receive the request. Communication interfaces 308 may include one or more interfaces configured to translate communications into particular native formats (e.g., enabling communications between disparate device types), generate interfaces to communicate with new devices, execute commands (e.g., via a application programming interfaces, remote procedural calls, etc.), generate interfaces to present content generated by machine-learning models 312 (e.g., such as webpages, images, infographics, etc.). Communication interfaces 308 may translate the request into a format that can be parsed by a management process of machine-learning models 312.

The request may include one or more features of the machine-learning model requested. If no such machine-learning model exists, communication network may select a set of communication sessions from communication session 316 and/or corresponding electronic health records of users of the selected communication session. The communication sessions may be selected based on the features included in the request. Examples of features may include, but are not limited to, an identification of a health system, insurance provider, an identification of a healthcare provider, an identification of a patient, demographic information associated with the patient, an identification of a practice area of the healthcare providers, purpose of the communication session (e.g., checkup, treatment, symptoms, diagnosis, diagnostics or testing, therapy, combinations thereof, and/or the like), an identification of a treatment being provided, an identification of symptoms experienced by a patient, an identification of diseases diagnosed by the healthcare provider, an identification of diseases that the patient is diagnosed with, and/or the like.

Communication network 120 may use the selected communication sessions from communication session 316 and corresponding electronic health records 320 to define a training dataset for a machine-learning model usable to satisfy the request (e.g., as previously described). Examples of machine-learning models may include, but are not limited to, deep learning networks, neural networks, transformers (such as generative pre-trained transformers (GPT), Bidirectional Encoder Representations from Transformers (BERTs), text-to-text-transfer-transformer (T5), etc.), classifier, variational autoencoders, generative adversarial networks (GANs), recurrent gated units (GRUs), combinations thereof, or the like. In some instances, the machine-learning model may be generative pre-trained transformer type model. In other instances, the machine-learning model may be an ensemble model comprised of one or more interconnected machine-learning models (e.g., of a same type or one or more types).

The machine-learning model may be trained using supervised learning, unsupervised learning, semi-supervised learning, transfer learning, metalearning, reinforcement learning, combinations thereof, or the like using the training dataset. The machine-learning model may be trained over a predetermined time interval, for predetermined quantity of iterations, and/or until one or more accuracy metrics are reached (e.g., such as, but not limited to, accuracy, precision, area under the curve, logarithmic loss, F1 score, a longest common subsequence (LCS) such as ROUGE-L, Bilingual evaluation Understudy (BLEU) mean absolute error, mean square error, or the like.

Client device 304 may transmit a request to generate a context using the selected machine-learning model. The request may be a natural language statement or question, a query, a command, a data object, an image, an audio segment, a video segment, combinations thereof, and/or the like that may indicate, to the machine-learning model, what context is to be generated (e.g., a dataset, a study, trends, a single variate analysis, a multivariate analysis, root cause analysis, statistical analysis, sentiment analysis (e.g., of treatments, healthcare providers, healthcare systems, etc.), fraud detection (e.g., fraud, waste, and/or abuse of the healthcare provider, communication network 120, healthcare system, and/or the like). The request may also include an identification of an output representation indicating a format of the output of the machine-learning model (e.g., text, images, infographics, video, audio segments, etc.). communication interfaces 308 may translate the request and pass it to the management process of machine-learning models 312.

The management process of machine-learning models 312 may execute the selected machine-learning model using a feature vector generated from the request. The feature vector may include an ordered sequence of features included in the request and/or derived from the request (e.g., such as features inferred from the request, features generated from interpolation or extrapolation, features retrieved from communication sessions 316 or EHR 320 that are associated with the request, combinations thereof, and/or the like). The ordered sequence of the feature vector may be based on the selected machine-learning model and/or the request. For example, the feature vector may be ordered according to time (or other dimension), a hierarchy of features to be included in the feature vector, weights assigned to the features to be included in the feature vector, a type associated with the features to be included in the feature vector (e.g., features of the request may be included before features derived from the request), combinations thereof, and/or the like.

The machine-learning model may execute using the feature vector to generate an output according to the request. The output may be dataset, text, audio segment, video segment, image, infographic, etc. For example, client device 304 may request a study of communication sessions of a healthcare system for which a particular treatment is offered or being provided. The machine-learning model may identify patients that have been provided and/or offered the treatment, communication sessions in which the treatment is offered and/or provided, communication sessions in which the treatment is discussed, etc. and generate a report indicating a sentiment of the treatment among patients (e.g., with quotes and/or excerpts from the communication sessions, etc.), an efficacy of the treatment, an indication of other treatments provided in place of the particular treatment, etc. The report may include text, charts, graphs, trends, etc. associated with the particular treatment. The machine-learning model may be configured to retrieve information associated with communication sessions, perform statistical analysis (e.g., multivariate, single variate, regression analysis, etc.), detect trends, interpolate data, extrapolate data, generate predictions (e.g., such as efficacy of treatments, conversational approaches such as a likelihood that a patient may respond to a given statement or request, determine a sentiment regarding a particular topic or treatment, combinations thereof, and/or the like.

Although shown as a component of the communication network, the machine-learning model may be instantiated, trained, and/or operated from any device such as, but not limited to, client device 304, a device associated with a healthcare provider, a device associated with healthcare system, etc. The training data used to train the machine-learning model may defined based on the location in which the machine-learning model will be trained and/or operated. Alternatively, or additionally the training data used to train the machine-learning model may defined based on the device requesting access to the machine-learning model.

For instance, if the machine-learning model is being trained and/or operated by the healthcare provider and/or the healthcare system of the healthcare provider, then the training dataset may be defined to include personal identifiable information (unless blind studies are to be performed) because the personal identifiable information of the records is already within the control of the healthcare provider and/or the healthcare system. If the machine-learning model is being trained and/or operated by a user or entity outside the healthcare provider and/or the healthcare system of the healthcare provider, then the training dataset may be defined using anonymized data (e.g., where personal identifiable information is removed) to prevent the trained machine-learning model from leaking personal identifiable information associated with patients and/or healthcare providers. Since the training dataset modified to remove personal identifiable information, the machine-learning model may not hallucinate or otherwise generate results that may be associated with an identity of a particular patient.

The output generated by the machine-learning model may be transmitted to client device 304 through communication interfaces 308. In some instances, the output may be used to execute a subsequent training iteration of the machine-learning model. Alternatively, or additionally, feedback provided by client device 308 may be used to execute a reinforcement learning iteration.

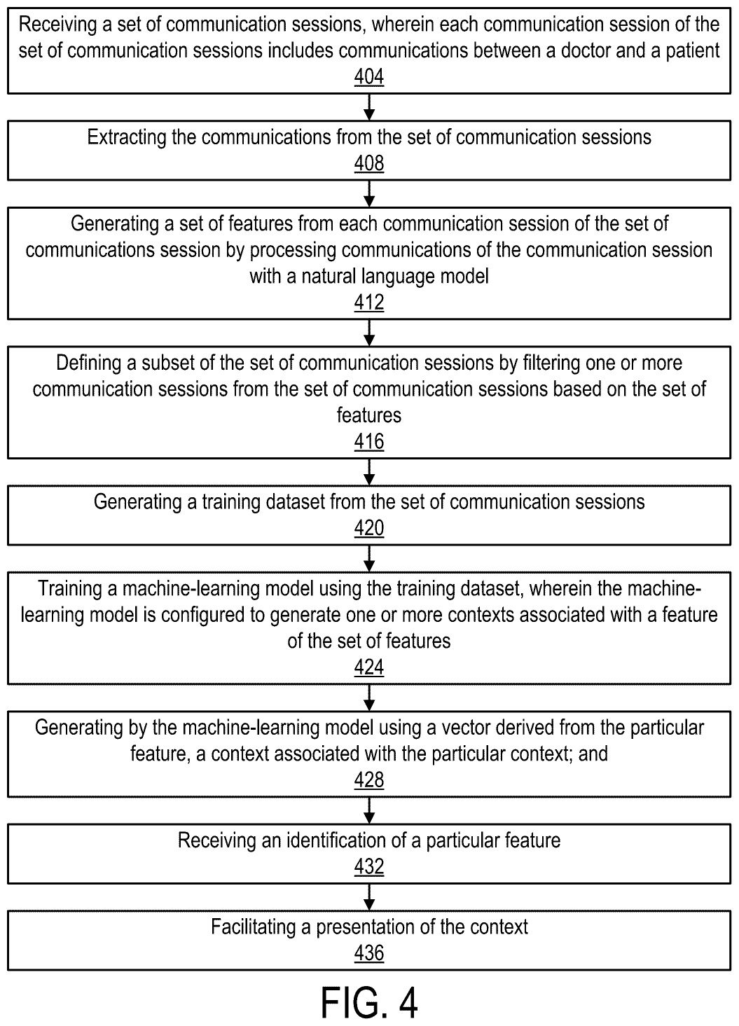

FIG. 4 illustrates a flowchart of an example process for generating a review environment of the communication network configured for a particular user device according to aspects of the present disclosure. At block 404, a computing device (e.g., a component of a communication network such as communication network 120 of FIG. 1, server in communication with a communication network, device in communication with one or more other devices, etc.) may receive a set of communication sessions. Each communication session may be a record of a telehealth session that includes communications between a healthcare provider and a patient. The computing device may receive the set of communication sessions from local or remote memory, one or more other instances of a communication network, combinations thereof, and/or the like. Each communication session may include a record of the telehealth session including, but not limited to, communications exchanged during the telehealth session (e.g., text, audio segments, video segments, combinations thereof, and/or the like), notes generated by the healthcare provider and/or the patient, metadata, data and/or media provided via a shared interface of the communication session, combinations thereof, and/or the like.

At block 408, the computing device may extract the communications from the set of communication sessions. In some instances, the computing device may extract the communications from each communication session of the set of communication sessions. In other instances, the computing device may extract particular communications from each communication session of the set of communication sessions or extract the communications from a particular subset of communication sessions of the set of communication session. The particular communications and/or the particular subset of communication sessions may be selected based on one or more parameters such as an identification of a meaning and/or intent of the communication, an identification of a particular healthcare system associated with the communication or communication session (e.g., such as a healthcare office, healthcare network, insurance provider, etc.), an identification of a healthcare provider practice area (e.g., general practitioner, psychology/psychiatry, dermatology, etc.) associated with the communication or communication session, an identification of a purpose of the healthcare session (e.g., checkup, treatment, symptoms, diagnosis, diagnostics or testing, therapy, combinations thereof, and/or the like) combinations thereof, and/or the like.

At block 412, the computing device may generate a set of features from each communication session of the set of communications session by processing the extracted communications with a natural language model. The extracted communications may include natural language text, speech, and/or gestures. The communication network may translate the communications into an alphanumeric representation (e.g., using a speech-to-text model for speech, an image classifier for gestures, etc.). The natural language model may generate features from the communications including, but not limited to, a meaning of communications, an intent of communications, an identification of communications that may be used to identify the patient or the healthcare provider, an identification of parts of speech and grammar, combinations thereof, and/or the like. The natural language model may then remove portions of the alphanumeric representation that may not be relevant or useful such as communications that do not correspond to a purpose of the communication session (e.g., small talk, social communications, etc.), articles and/or other grammatical artifacts, etc. The natural language model may also normalize the remaining alphanumeric representation (e.g., such as by removing unnecessary portions of the alphanumeric representation such as articles and/or punctuation, replacing words with unconjugated variations, reducing words variation by replacing synonyms or similarly defined words with a particularly selected word, etc.). The normalized alphanumeric representation may be stored in a first data object.

In some examples, the computing device may process other data of a communication session such as, but not limited to, data associated with devices operated by the healthcare provider and/or the patient, metadata, objects transmitted via a shared interface of the communication session, combinations thereof, and/or the like. The other data may be processed by one or more machine-learning models, the natural language model, one or more algorithms, user input, any of the aforementioned processing techniques, combinations thereof, and/or the like. The processed other data may be stored in a second data object that may be combined with the first data object or stored in association with the first data object.

At block 416, the computing device may define a subset of the set of communication sessions by filtering, from the first data object and/or the second data object, data of one or more communication sessions from the set of communication sessions based on the set of features. In some instances, the computing device may define a subset of the set of communication sessions when the computing devices extracts communications from each of the communication sessions. In those instances, if the computing device extracts communications from a subset of the communication sessions, then block 416 may be skipped). In other instances, the computing device may define a subset of the set of communication sessions in addition to the selective extraction of communications as described above. The features may include, but are not limited to, a practice area of the healthcare provider associated with the communication session, purpose of the communication session (e.g., checkup, treatment, symptoms, diagnosis, diagnostics or testing, therapy, combinations thereof, and/or the like), an identification of a diagnosis, an identification of a healthcare system, demographic information associated with the patient and/or healthcare provider, a frequency of communication sessions between the healthcare provider and the patient, a frequency of communication sessions associated with the healthcare provider, a frequency of communication sessions associated with the patient, combinations thereof, and/or the like.

At block 420, the computing device may generate a training dataset from the subset of the set of communication sessions. In some examples, the computing device may evaluate the training dataset to determine if additional data is needed for the training dataset to be usable. For example, when the training dataset is too small or is limited (e.g., particular data is over-represented in the training data and/or particular data is under-represented in the training data, etc.), additional data may be added to the training dataset. The additional data may include manually generated data, procedurally generated data, combinations thereof, and/or the like. Metadata may also be added to the training data to enable particular types of learning algorithm. For instance, for supervised learning, labels (as metadata) may be added to the training data. The labels may be generated manually (e.g., via user input, etc.), by an already trained instance of the machine-learning model, by a generative adversarial network, combinations thereof, and/or the like.

At block 424, the computing device may train a machine-learning model using the training dataset. The machine-learning model may be a generative machine-learning model generate one or more contexts associated with a feature of the set of features. A context may correspond to a trend, query response, analytic, efficacy of a treatment, single variate analysis, multi variate analysis, modification (e.g., to the computing device, a communication network, a communication session, etc.), a recommended improvement, a classification, a sentiment, an intent, combination thereof, and/or the like. Examples of machine-learning models may include, but are not limited to, deep learning networks, neural networks, transformers (such as generative pre-trained transformers (GPT), Bidirectional Encoder Representations from Transformers (BERTs), text-to-text-transfer-transformer (T5), etc.), classifier, variational autoencoders, generative adversarial networks (GANs), recurrent gated units (GRUs), combinations thereof, or the like. In some instances, the machine-learning model may be generative pre-trained transformer type model. In other instances, the machine-learning model may be an ensemble model comprised of one or more interconnected machine-learning models (e.g., of a same type or of one or more types).

The machine-learning model may be trained using supervised learning, unsupervised learning, semi-supervised learning, transfer learning, metalearning, reinforcement learning, combinations thereof, or the like using the training dataset. The machine-learning model may be trained over a predetermined time interval, for predetermined quantity of iterations, and/or until one or more accuracy metrics are reached (e.g., such as, but not limited to, accuracy, precision, area under the curve, logarithmic loss, F1 score, a longest common subsequence (LCS) such as ROUGE-L, Bilingual evaluation Understudy (BLEU) mean absolute error, mean square error, or the like.

At block 428, the computing device may receive a request associated with a particular feature. The request may include a query, a natural language statement, a command, an image, an audio segment, a video segment, combinations thereof, and/or the like. The computing device may generate a feature vector from the request and based on the feature. The feature vector may include the request (e.g., in a same representation as received, translated into a particular representation, translated into a particular sequence, and/or the like).

At block 432, the computing device may generate, using the machine-learning model and a feature vector derived from the request, a context associated with the particular feature. For example, the request may correspond to a study associated with a treatment protocol provided by the healthcare provider to one or more patients. The context may include trends, analytics, single variate and/or multivariate analysis, combinations thereof, and/or the like providing characteristics of the treatment protocol such as efficacy of the treatment protocol, an identification of outliers in the treatment (e.g., such as new or rarely identified side effects, etc.), identification of patient sentiment of the treatment protocol, an identification of recommended follow up studies and/or treatments, root cause analysis, charts, graphs, infographics, combinations thereof, and/or the like.

At block 436, the computing device may facilitate a presentation of the context. The presentation of the context may include presenting the context via a graphical user interface, a video segment, an audio segment (e.g., using narration derived from a text-to-speech model and/or a user), combinations thereof, and/or the like. In some instances, the context may be used to modify the computing device, subsequent communication sessions facilitated by a communication network, the machine-learning model (e.g., via reinforcement learning, etc.), combinations thereof, and/or the like.

The process may then return to block 428 and wait until a subsequent request is received. Blocks 404-424 may be executed once to train the machine-learning model. Once trained blocks 428-436 may be executed any number of times. In some instance, a context generated by the machine-learning model may cause the machine-learning model to initiate a retraining iteration and/or a reinforcement learning. For example, an analysis of communication sessions may cause the training dataset to be updated (e.g., using the generated analysis). The updated training dataset may be used to update the machine-learning model (e.g., through reinforcement learning, a training iteration, etc.) or used to train a subsequent instance of the machine-learning model. Each execution of the machine-learning model may further train the machine-learning model causing iterative improvements of the machine-learning model. Alternatively or additionally, the machine-learning model may cause the computing device (or communication network) to be updated thereby causing iterative improvements to subsequent communication sessions based on machine-learning model processing previous communication sessions (e.g., the subset of the set of communications session). Since the processes of FIG. 4 self-select communications through filtering (e.g., at block 416), the updates to the machine-learning model, the computing device, and/or the communication network may also be self-selected by the machine-learning model based on subset of the set of communication session and the criteria used in the filtering at block 416.

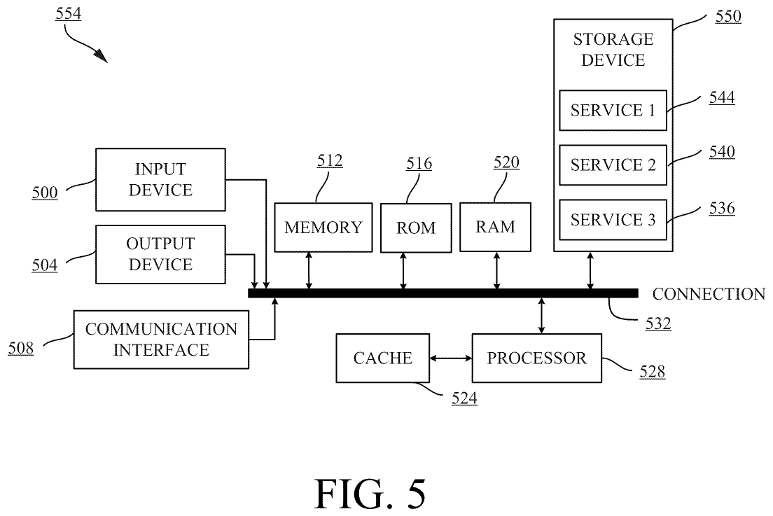

FIG. 5 illustrates an example computing device according to aspects of the present disclosure. For example, computing device 500 can implement any of the systems or methods described herein. In some instances, computing device 500 may be a component of or included within a media device. The components of computing device 500 are shown in electrical communication with each other using connection 506, such as a bus. The example computing device architecture 500 includes a processor (e.g., CPU, processor, or the like) 504 and connection 506 (e.g., such as a bus, or the like) that is configured to couple components of computing device 500 such as, but not limited to, memory 520, read only memory (ROM) 518, random access memory (RAM) 516, and/or storage device 508, to processing unit 510.

Computing device 500 can include a cache 502 of high-speed memory connected directly with, in close proximity to, or integrated within processor 504. Computing device 500 can copy data from memory 520 and/or storage device 508 to cache 502 for quicker access by processor 504. In this way, cache 502 may provide a performance boost that avoids delays while processor 504 waits for data. Alternatively, processor 504 may access data directly from memory 520, ROM 517, RAM 516, and/or storage device 508. Memory 520 can include multiple types of homogenous or heterogeneous memory (e.g., such as, but not limited to, magnetic, optical, solid-state, etc.).

Storage device 508 may include one or more non-transitory computer-readable media such as volatile and/or non-volatile memories. A non-transitory computer-readable medium can store instructions and/or data accessible by computing device 500. Non-transitory computer-readable media can include, but is not limited to magnetic cassettes, hard-disk drives (HDD), flash memory, solid state memory devices, digital versatile disks, cartridges, compact discs, random access memories (RAMs) 525, read only memory (ROM) 520, combinations thereof, or the like.

Storage device 508, may store one or more services, such as service 1 510, service 2 512, and service 3 514, that are executable by processor 504 and/or other electronic hardware. The one or more services include instructions executable by processor 504 to: perform operations such as any of the techniques, steps, processes, blocks, and/or operations described herein; control the operations of a device in communication with computing device 500; control the operations of processing unit 510 and/or any special-purpose processors; combinations therefor; or the like. Processor 504 may be a system on a chip (SOC) that includes one or more cores or processors, a bus, memories, clock, memory controller, cache, other processor components, and/or the like. A multi-core processor may be symmetric or asymmetric.

Computing device 500 may include one or more input devices 522 that may represent any number of input mechanisms, such as a microphone, a touch-sensitive screen for graphical input, keyboard, mouse, motion input, speech, media devices, sensors, combinations thereof, or the like. Computing device 500 may include one or more output devices 524 that output data to a user. Such output devices 524 may include, but are not limited to, a media device, projector, television, speakers, combinations thereof, or the like. In some instances, multimodal computing devices can enable a user to provide multiple types of input to communicate with computing device 500. Communications interface 526 may be configured to manage user input and computing device output. Communications interface 526 may also be configured to managing communications with remote devices (e.g., establishing connection, receiving/transmitting communications, etc.) over one or more communication protocols and/or over one or more communication media (e.g., wired, wireless, etc.).

Computing device 500 is not limited to the components as shown in FIG. 5. Computing device 500 may include other components not shown and/or components shown may be omitted.

As used below, any reference to a series of examples is to be understood as a reference to each of those examples disjunctively (e.g., “Examples 1-4” is to be understood as “Examples 1, 2, 3, or 4”).

Example 1 is a computer-implemented method comprising: receiving a set of communication sessions, wherein each communication session of the set of communication sessions includes communications between a doctor and a patient; extracting the communications from the set of communication sessions; generating a set of features from each communication session of the set of communications session by processing communications of the communication session with a natural language model; defining a subset of the set of communication sessions by filtering one or more communication sessions from the set of communication sessions based on the set of features; generating a training dataset from the set of communication sessions; training a machine-learning model using the training dataset, wherein the machine-learning model is configured to generate one or more contexts associated with a feature of the set of features; receiving a request associated with a particular feature; generating by the machine-learning model using a feature vector derived from the request, a context associated with the particular feature; and facilitating a presentation of the context.

Example 2 is the computer-implemented method of any of example(s) 2-7, wherein processing communications of the communication session with a natural language model includes classifying communications according to a contextual hierarchy.

Example 3 is the computer-implemented method of any of example(s) 1-2 and 4-7, wherein processing communications of the communication session with a natural language model includes filtering personal identifiable information from the communications.

Example 4 is the computer-implemented method of any of example(s) 1-3 and 5-7, wherein the machine-learning model is generative transformer model.

Example 5 is the computer-implemented method of any of example(s) 1-4 and 6-7, wherein the context identifies a relationship between patients and the particular feature.

Example 6 is the computer-implemented method of any of example(s) 1-5 and 7, wherein the context includes a representation of the particular feature that is customized for a portion of patients.

Example 7 is the computer-implemented method of any of example(s) 1-6, wherein the context indicates an efficacy of a treatment associated with the particular feature.

Example 8 is a system comprising one or more processors; and a non-transitory computer-readable medium storing instructions that when executed by the one or more processors cause the one or more processors to perform operations of any of example(s) 1-7.

Example 9 is non-transitory computer-readable medium storing instructions that when executed by one or more processors cause the one or more processors to perform operations of any of example(s) 1-7.

The term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored in a form that excludes carrier waves and/or electronic signals. Examples of a non-transitory medium may include, but are not limited to, a magnetic disk or tape, optical storage media such as compact disk (CD) or digital versatile disk (DVD), flash memory, memory, or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, or the like.

Some portions of this description describe examples in terms of algorithms and symbolic representations of operations on information. These operations, while described functionally, computationally, or logically, may be implemented by computer programs or equivalent electrical circuits, microcode, or the like. Furthermore, arrangements of operations may be referred to as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In some examples, a software module can be implemented with a computer-readable medium storing computer program code, which can be executed by a processor for performing any or all of the steps, operations, or processes described.

Some examples may relate to an apparatus or system for performing any or all of the steps, operations, or processes described. The apparatus or system may be specially constructed for the required purposes, and/or it may comprise a general-purpose computing device selectively activated or reconfigured by a computer program stored in memory of computing device. The memory may be or include a non-transitory, tangible computer readable storage medium, or any type of media suitable for storing electronic instructions, which may be coupled to a bus. Furthermore, any computing systems referred to in the specification may include a single processor or multiple processors.

While the present subject matter has been described in detail with respect to specific examples, it will be appreciated that those skilled in the art, upon attaining an understanding of the foregoing, may readily produce alterations to, variations of, and equivalents to such embodiments. Numerous specific details are set forth herein to provide a thorough understanding of the claimed subject matter. However, those skilled in the art will understand that the claimed subject matter may be practiced without these specific details. In other instances, methods, apparatuses, or systems that would be known by one of ordinary skill have not been described in detail so as not to obscure claimed subject matter. Accordingly, the present disclosure has been presented for purposes of example rather than limitation, and does not preclude the inclusion of such modifications, variations, and/or additions to the present subject matter as would be readily apparent to one of ordinary skill in the art.

For clarity of explanation, in some instances the present disclosure may be presented as including individual functional blocks including functional blocks comprising devices, device components, steps or routines in a method embodied in software, or combinations of hardware and software. Additional functional blocks may be used other than those shown in the figures and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Individual examples may be described herein as a process or method which may be depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed but may have additional steps not shown. A process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general-purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc.

Devices implementing the methods and systems described herein can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. The program code may be executed by a processor, which may include one or more processors, such as, but not limited to, one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A processor may be a microprocessor; conventional processor, controller, microcontroller, state machine, or the like. A processor may also be implemented as a combination of computing components (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. Functionality described herein also can be embodied in peripherals or add-in cards. Such functionality can also be implemented on a circuit board among different chips or different processes executing in a single device, by way of further example.

In the foregoing description, aspects of the disclosure are described with reference to specific examples thereof, but those skilled in the art will recognize that the disclosure is not limited thereto. Thus, while illustrative examples of the disclosure have been described in detail herein, it is to be understood that the inventive concepts may be otherwise variously embodied and employed, and that the appended claims are intended to be construed to include such variations. Various features and aspects of the above-described disclosure may be used individually or in any combination. Further, examples can be utilized in any number of environments and applications beyond those described herein without departing from the broader spirit and scope of the disclosure. The disclosure and figures are, accordingly, to be regarded as illustrative rather than restrictive.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

Unless specifically stated otherwise, it is appreciated that throughout this specification discussions utilizing terms such as “processing,” “computing,” “calculating,” “determining,” and “identifying” or the like refer to actions or processes of a computing device, such as one or more computers or a similar electronic computing device or devices, that manipulate or transform data represented as physical electronic or magnetic quantities within memories, registers, or other information storage devices, transmission devices, or media devices of the computing platform. The use of “adapted to” or “configured to” herein is meant as open and inclusive language that does not foreclose devices adapted to or configured to perform additional tasks or steps. Additionally, the use of “based on” is meant to be open and inclusive, in that a process, step, calculation, or other action “based on” one or more recited conditions or values may, in practice, be based on additional conditions or values beyond those recited. Headings, lists, and numbering included herein are for ease of explanation only and are not meant to be limiting.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claim.

1. A computer-implemented method comprising:
receiving a set of communication sessions, wherein each communication session of the set of communication sessions includes communications between a doctor and a patient;
extracting the communications from the set of communication sessions;
generating a set of features from each communication session of the set of communications session by processing communications of the communication session with a natural language model;
defining a subset of the set of communication sessions by filtering one or more communication sessions from the set of communication sessions based on the set of features;
generating a training dataset from the set of communication sessions;
training a machine-learning model using the training dataset, wherein the machine-learning model is configured to generate one or more contexts associated with a feature of the set of features;
receiving a request associated with a particular feature;
generating by the machine-learning model using a feature vector derived from the request, a context associated with the particular feature; and
facilitating a presentation of the context.

2. The computer-implemented method of claim 1, wherein processing communications of the communication session with a natural language model includes classifying communications according to a contextual hierarchy.

3. The computer-implemented method of claim 1, wherein processing communications of the communication session with a natural language model includes filtering personal identifiable information from the communications.

4. The computer-implemented method of claim 1, wherein the machine-learning model is generative transformer model.

5. The computer-implemented method of claim 1, wherein the context identifies a relationship between patients and the particular feature.

6. The computer-implemented method of claim 1, wherein the context includes a representation of the particular feature that is customized for a portion of patients.

7. The computer-implemented method of claim 1, wherein the context indicates an efficacy of a treatment associated with the particular feature.

8. A system comprising:
one or more processors and a non-transitory computer-readable medium storing instructions that when executed by the one or more processors cause the one or more processors to perform operations that include:
receiving a set of communication sessions, wherein each communication session of the set of communication sessions includes communications between a doctor and a patient;
extracting the communications from the set of communication sessions;
generating a set of features from each communication session of the set of communications session by processing communications of the communication session with a natural language model;
defining a subset of the set of communication sessions by filtering one or more communication sessions from the set of communication sessions based on the set of features;
generating a training dataset from the set of communication sessions;
training a machine-learning model using the training dataset, wherein the machine-learning model is configured to generate one or more contexts associated with a feature of the set of features;
receiving a request associated with a particular feature;
generating by the machine-learning model using a feature vector derived from the request, a context associated with the particular feature; and
facilitating a presentation of the context.

9. The system of claim 8, wherein processing communications of the communication session with a natural language model includes classifying communications according to a contextual hierarchy.

10. The system of claim 8, wherein processing communications of the communication session with a natural language model includes filtering personal identifiable information from the communications.

11. The system of claim 8, wherein the machine-learning model is generative transformer model.

12. The system of claim 8, wherein the context identifies a relationship between patients and the particular feature.

13. The system of claim 8, wherein the context includes a representation of the particular feature that is customized for a portion of patients.

14. The system of claim 8, wherein the context indicates an efficacy of a treatment associated with the particular feature.

15. A non-transitory computer-readable medium storing instructions that when executed by one or more processors cause the one or more processors to perform operations that include:
receiving a set of communication sessions, wherein each communication session of the set of communication sessions includes communications between a doctor and a patient;
extracting the communications from the set of communication sessions;
generating a set of features from each communication session of the set of communications session by processing communications of the communication session with a natural language model;
defining a subset of the set of communication sessions by filtering one or more communication sessions from the set of communication sessions based on the set of features;
generating a training dataset from the set of communication sessions;
training a machine-learning model using the training dataset, wherein the machine-learning model is configured to generate one or more contexts associated with a feature of the set of features;
receiving a request associated with a particular feature;
generating by the machine-learning model using a feature vector derived from the request, a context associated with the particular feature; and
facilitating a presentation of the context.

16. The non-transitory computer-readable medium of claim 15, wherein processing communications of the communication session with a natural language model includes filtering personal identifiable information from the communications.

17. The non-transitory computer-readable medium of claim 15, wherein the machine-learning model is generative transformer model.

18. The non-transitory computer-readable medium of claim 15, wherein the context identifies a relationship between patients and the particular feature.

19. The non-transitory computer-readable medium of claim 15, wherein the context includes a representation of the particular feature that is customized for a portion of patients.

20. The non-transitory computer-readable medium of claim 15, wherein the context indicates an efficacy of a treatment associated with the particular feature.
